(19) United States
(12) Patent Application Publication
(10) Pub. No.: US 2025/0303132 A1
(43) Pub. Date: Oct. 2, 2025

(54) MICRONEEDLE DEVICE

(71) Applicant: SHISEIDO COMPANY, LTD., Chuo-ku, Tokyo (JP)

(72) Inventors: Kuan Ting LIN, Tokyo (JP); Kazuyuki MIYAZAWA, Tokyo (JP); Yukiko KOBAYASHI, Tokyo (JP)

(73) Assignee: SHISEIDO COMPANY, LTD., Chuo-ku, Tokyo (JP)

(21) Appl. No.: 18/864,212

(22) PCT Filed: Jun. 9, 2022
(86) PCT No.: PCT/JP2022/023352

§ 371 Date: Nov. 8, 2024

(51) Int. Cl. A61M 37/00

(52) U.S. Cl. CPC A61M 37/0015; A61M 2037/0023; A61M 2037/0046; A61M 2037/0061

(57) ABSTRACT

To provide a novel microneedle device which reduces the hassle of hydrating the skin in advance. A microneedle device 100 comprises an adhesive layer 1 and a microneedle sheet layer 10, wherein the adhesive layer 1 has one or more openings 1a, the microneedle sheet layer 10 has a substrate 2 and a plurality of microneedles 3 which protrude from the substrate 2, the substrate 2 and the microneedles 3 are formed from a material which dissolves or swells in vivo, and the microneedle sheet layer 10 is arranged so as to at least partially overlap the openings la of the adhesive layer 1.

### TECHNICAL FIELD

The present invention relates to a microneedle device.

### BACKGROUND ART

Beautification technology continues to advance day by day.

“Microneedle technology” is one of beautification technologies which have been attracting attention in recent years. In microneedle technology in the beauty industry, microneedles are formed by solidifying biodegradable ingredients such as hyaluronic acid into microneedle shapes and attached to the skin, whereby the ingredients such as the hyaluronic acid penetrate the skin. More specifically, a microneedle sheet comprising microneedles is attached to the skin with, for example, an adhesive tape, whereby the microneedles pierce the skin. Further, moisture in the skin diffuses into the microneedles, causing the needle portions inserted into the skin to swell and then dissolve. As a result, the biodegradable ingredients such as hyaluronic acid penetrate into the skin and exert a cosmetic effect.

Patent Literature 1 discloses, as a novel beautification method for improving blood circulation in the entire face to brighten the color tone of the entire face, a beautification method wherein the color tone of the entire face is brightened by attaching a microneedle sheet comprising a plurality of microneedles to a part of the face.

Furthermore, Patent Literature 2 discloses a microneedle patch which is quickly absorbed into the body and, as a method for the use thereof, a method (excluding medical procedures) of rapid dissolution of a microneedle array, wherein moisture is supplied from a back surface of a microneedle array comprising a substrate having a thickness of 500 μm or less, using a water-swellable polymer as a primary material, and the moisture swells the microneedle array.

Patent Literature 3 discloses a method in which an interfacial electrodynamic transmission system is used. More specifically, the method of Patent Literature 3 is a method wherein a medicament is delivered to a treatment site beneath an electrically-resistant layer of skin of an individual, the method comprising the steps of bringing a plurality of microneedles for penetrating the electrically-resistant layer of the skin of the individual into contact with the skin of the individual, and interfacially-driving the medicament or the medicament and an electrical carrier for the drug into the treatment site via the microneedles while bypassing the electrically resistant layer of the skin of the individual.

### CITATION LIST

### Patent Literature

PTL 1: Japanese Unexamined Patent Publication (Kokai) No. 2020-164432

PTL 2: Japanese Unexamined Patent Publication (Kokai) No. 2013-075165

PTL 3: WO 2007/035710

### SUMMARY OF INVENTION

### Technical Problem

When using a microneedle technology, in order for beauty ingredients or the like constituting the microneedle sheet layer (and in particular, the microneedles) to penetrate the skin, it is necessary to stably hold the microneedle sheet layer on the skin surface in a state in which needle portions thereof are inserted into the skin. Conventionally, an adhesive layer such as adhesive tape which has a larger area than the area of the back surface of the microneedle sheet layer (the surface opposite to the needles) is arranged on the back surface of the microneedle sheet layer, and due to the adhesive force of the adhesive layer, the microneedle sheet layer is attached to the skin surface in a state in which the needle portions are inserted into the skin.

However, when the entire back of the microneedle sheet layer is in close contact with the adhesive layer, depending on the constituent material of the adhesive layer, a component permeating from the adhesive layer may affect the performance of the microneedle sheet layer. Thus, there is room for improvement in the use of microneedle technology.

The present invention seeks to improve the above circumstances, and an object thereof is to provide a novel microneedle device with which the influence of the adhesive layer, which is in contact with the back of the microneedle sheet layer, on the performance of the microneedle sheet layer can be suppressed.

### Solution to Problem

The present invention, which can achieve the above object, is as described below.

### <Aspect 1>

A microneedle device, comprising an adhesive layer and a microneedle sheet layer,
wherein the adhesive layer has one or more openings,
wherein the microneedle sheet layer has a substrate and a plurality of microneedles which protrude from the substrate,
wherein the substrate and the microneedles are formed from a material which dissolves or swells in vivo, and
wherein the microneedle sheet layer is arranged so as to at least partially overlap the openings of the adhesive layer.

### <Aspect 2>

The microneedle device according to Aspect 1, further comprising an electrode sheet layer on a surface of the adhesive layer on a side opposite the microneedles.

### <Aspect 3>

The microneedle device according to Aspect 1 or 2, wherein the microneedle sheet layer is arranged so as to overlap the openings of the adhesive layer or inside the
openings.

### <Aspect 4>

The microneedle device according to any one of Aspects 1 to 3, further comprising a support layer, wherein the support layer is arranged in the openings of the adhesive layer, and
wherein the microneedle sheet layer is supported on the support layer.

### <Aspect 5>

The microneedle device according to any one of Aspects 1 to 4, wherein the material which dissolves or swells in vivo is hyaluronic acid.

### <Aspect 6>

The microneedle device according to any one of Aspects 1 to 5, for skincare use.

### <Aspect 7>

A beautification method using the microneedle device according to any one of Aspects 1 to 6, the method comprising the step of:
attaching and adhering the microneedle side of the microneedle device to skin,
whereby at least a part of a constituent material of the microneedles penetrates the skin.

### <Aspect 8>

A beautification method using the microneedle device according to any one of Aspects 2 to 6, the method comprising the steps of:

attaching and adhering the microneedle side of the microneedle device to skin,
whereby at least a part of a constituent material of the microneedles penetrates the skin, and
applying a pulsed current to the skin via the electrode sheet layer.

### Advantageous Effects of Invention

According to the present invention, there can be provided a novel microneedle device with which the hassle of hydrating the skin in advance can be reduced and with which the needle portions inserted in the skin can efficiently dissolve.

### BRIEF DESCRIPTION OF DRAWINGS

FIG. 1 is a schematic cross-sectional view showing an example of the microneedle device of the present invention.

FIG. 2 is a plan view showing several embodiments of adhesive layers having one or more openings.

FIG. 3 is a cross-sectional view showing an embodiment of the microneedle device of the present invention including an electrode sheet layer.

FIG. 4 is a schematic view showing an embodiment of an electrode sheet layer according to the present invention.

FIG. 5 is a schematic view showing an embodiment of an electrode sheet layer according to the present invention.

FIG. 6 is a cross-sectional view showing an embodiment of the microneedle device of the present invention including a support layer.

FIG. 7 is a pattern diagram for detailing an embodiment of a first beautification method of the present invention.

FIG. 8 is a pattern diagram for detailing an embodiment of a second beautification method of the present invention.

FIG. 9 is a view showing results of hyaluronic acid penetration strength of Examples 1 and 2 and Comparative Example 1.

FIG. 10 is a view showing results of hyaluronic acid penetration strength of Example 2 and Comparative Example 2.

### DESCRIPTION OF EMBODIMENTS

The embodiments of the present invention will be described in detail below with reference to the drawings. Note that in order to facilitate understanding, in the drawings, identical or corresponding portions have been assigned the same reference signs, and duplicate descriptions thereof have been omitted. Furthermore, the present invention is not limited to the following embodiments, and various modifications can be made within the scope of the spirit of the invention.

### Microneedle Device

The microneedle device of the present invention comprises:
an adhesive layer and a microneedle sheet layer,
wherein the adhesive layer has one or more openings,
wherein the microneedle sheet layer has a substrate and a plurality of microneedles which protrude from the substrate,
wherein the substrate and the microneedles are formed from a material which dissolves or swells in vivo, and
wherein the microneedle sheet layer is arranged so as to at least partially overlap the openings of the adhesive layer.

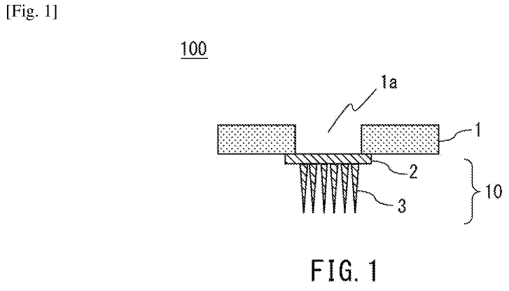

FIG. 1 is a schematic cross-sectional view showing an example of the microneedle device of the present invention.

The microneedle device 100 of the present invention shown in FIG. 1 comprises an adhesive layer 1 and a microneedle sheet layer 10. In the microneedle device 100, the adhesive layer 1 has one or more openings la. Furthermore, the microneedle sheet layer 10 has a substrate 2 and a plurality of microneedles 3, which protrude from the substrate 2. Furthermore, as shown in FIG. 1, the microneedle sheet layer 10 is arranged so as to at least partially overlap the openings la of the adhesive layer 1.

When the entire back surface of the microneedle sheet layer is in contact with the adhesive layer, a component permeating from the adhesive layer may affect the performance of the microneedle sheet layer depending on the constituent material of the adhesive layer.

Specifically, for example, when a hydrogel layer having a high moisture content is used as the adhesive layer, the hydrogel layer retains skin moisture and/or hydrates the skin in combination with the role of attaching and holding the microneedle sheet layer on the skin, thereby promoting swelling and/or dissolution of the material forming the microneedles in the skin. However, in such a case, if the entire back surface of the microneedle sheet layer is in contact with the adhesive layer, the moisture in the hydrogel layer may cause the microneedle sheet layer to swell and/or dissolve before attachment of the microneedle sheet layer to the skin.

In connection thereto, according to the present invention, the adhesive layer has one or more openings and the microneedle sheet at least partially overlaps the openings of the adhesive layer, whereby a component permeating from the adhesive layer, for example, water permeating from the hydrogel layer as an adhesive layer, is prevented from influencing the performance of the microneedle sheet layer.

Each of the members which may constitute the microneedle device of the present invention will be described in detail below.

### <Adhesive Layer>

In the present invention, the adhesive layer may be, for example, one which has holding power when attached to the skin and which does not cause pain when peeled off. The adhesive layer may be a layer in which an adhesive component is provided on one surface of the substrate of the adhesive layer by application, impregnation, adhesion, casting, etc.

The adhesive component is not particularly limited, and examples thereof include various water-soluble polymers, oil-soluble polymers, and gel agents obtained by partial modification thereof using a means such as cross-linking. More specifically, the adhesive component may be but is not limited to, for example, a hydrogel.

The adhesive layer contains a conductive material such as conductive particles, whereby conductivity is imparted thereto. Examples of the conductive material such as conductive particles include carbon, graphite, carbon nanotubes, organic conductive substances such as PEDOT-PSS, and metals such as copper, aluminum, and silver. When the adhesive layer has conductivity, a pulsed current can be suitably applied to the skin using it together with, for example, an electrode sheet layer, which will be described later.

As described above, when a hydrogel layer is used as the adhesive layer, the hydrogel layer retains skin moisture and/or hydrates the skin in combination with the role of attaching and holding the microneedle sheet layer on the skin, thereby promoting swelling and/or dissolution of the material forming the microneedles in the skin.

Furthermore, since the hydrogel has conductivity, when, for example, an electrode sheet layer, which will be described later, is used together, application of a pulsed current to the skin can suitably be performed.

An adhesive layer in which a hydrogel is used will illustratively be described below.

### (Hydrogel)

As used herein, “hydrogel” is a general term for gel-like hydrophilic polymers containing moisture in the interior thereof. More specifically, examples of the hydrogel include gels containing a natural polymer such as agar, gelatin, agarose, xanthan gum, gellan gum, sclerotium gum, gum arabic, gum tragacanth, karaya gum, cellulose gum, tamarind gum, guar gum, locust bean gum, glucomannan, chitosan, carrageenan, quince seed, galactan, mannan, starch, dextrin, curdran, casein, pectin, collagen, fibrin, peptide, a chondroitin sulfate such as sodium chondroitin sulfate, hyaluronic acid (mucopolysaccharide), a hyaluronate such as sodium hyaluronate, alginic acid, an alginate such as sodium alginate or calcium alginate, or a derivative thereof; gels containing a cellulose derivative such as methyl cellulose, hydroxymethyl cellulose, hydroxyethyl cellulose, hydroxypropyl cellulose, hydroxypropylmethyl cellulose, carboxymethyl cellulose, or a salt thereof; gels containing a poly(meth)acrylic acid such as polyacrylic acid or polymethacrylic acid, an acrylic acid/alkyl methacrylate copolymer or a salt thereof; gels containing a synthetic polymer such as polyvinyl alcohol, polyhydroxyethyl methacrylate, polyacrylamide, poly(N-isopropylacrylamide), polyvinylpyrrolidone, polystyrene sulfonic acid, polyethylene glycol, a carboxyvinyl polymer, an alkyl-modified carboxyvinyl polymer, a maleic anhydride copolymer, a polyalkylene oxide resin, an N-vinylacetamide crosslinked product, an acrylamide crosslinked product, or a starch/acrylate graft copolymer crosslinked product; silicone hydrogels; interpenetrating network hydrogels and semi-interpenetrating network hydrogels; and mixtures of two or more thereof. However, the hydrogel is not limited thereto.

### (Substrate of Adhesive Layer)

The substrate of the adhesive layer is not particularly limited, and may be, for example, a woven fabric, a non-woven fabric, a porous sheet, a mesh, or a film.

Furthermore, as the substrate of the adhesive layer, for example, an electrode sheet layer, which will be described later, may be used. In this case, due to the adhesive layer, a pulsed current with a predetermined frequency can be applied to the skin, which enables collagen production, moisturization, and improvement of wrinkles in the skin.

### (Openings of Adhesive Layer)

In the microneedle device of the present invention, the adhesive layer has one or more openings. The openings of the adhesive layer are for reducing the contact area between the microneedle sheet layer and the adhesive layer, or for preventing contact between the microneedle sheet layer and the adhesive layer. Thus, the number of openings is not particularly limited as long as it is one or more, and may be appropriately adjusted according to the size of the area of the target microneedle device, the size of the microneedle sheet layer, etc.

For example, the adhesive layer may have one or more, two or more, three or more, four or more, or five or more openings, and may have ten or less, nine or less, eight or less, seven or less, six or less, or five or less openings. The shapes of the openings are not particularly limited, and may be appropriately adjusted according to the shape of the target microneedle device or the shape of the microneedle sheet layer. Furthermore, the sizes and positions of the openings are not particularly limited, and may be appropriately adjusted according to the size of the area of the target microneedle device, the arrangement location of the microneedle sheet layer, etc.

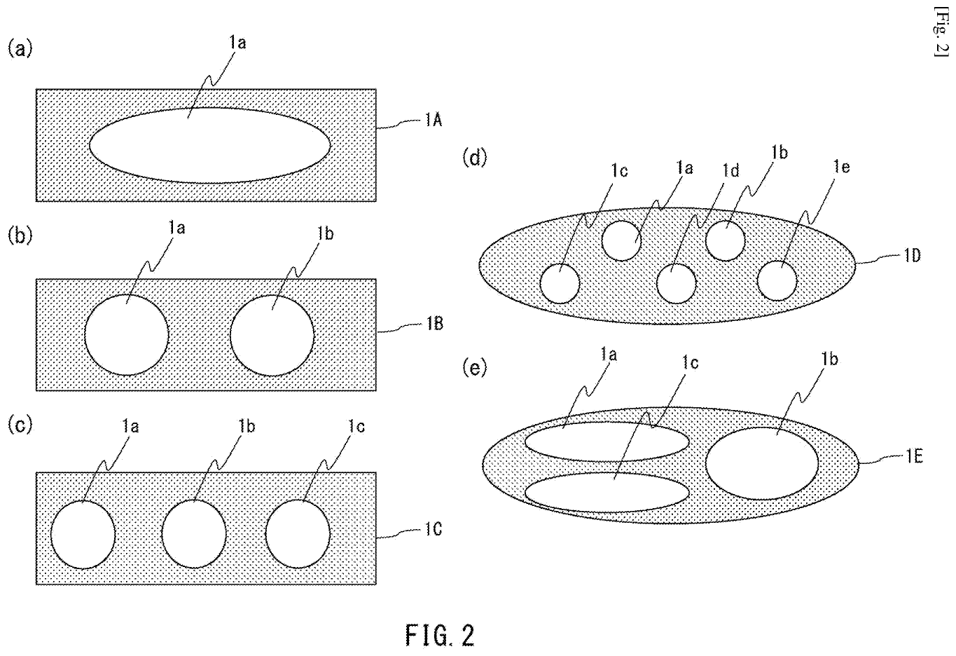

FIG. 2 is a plan view showing several embodiments of an adhesive layer having one or more openings. More specifically, the adhesive layer 1A shown in FIG. 2(a) has one opening 1a. The adhesive layer 1B shown in FIG. 2(b) has two openings 1a and 1b. The adhesive layer 1C shown in FIG. 2(c) has three openings 1a, 1b, and 1c. The adhesive layer 1C shown in FIG. 2(d) has five openings 1a, 1b, 1c, 1d, and 1e. The adhesive layer 1E shown in FIG. 2(e) has three openings 1a, 1b, and 1c. As shown in FIGS. 2(a) and 2(e), the numbers, shapes, sizes, positions, etc., of the openings present in the adhesive layer are not particularly limited.

### (Moisture Content)

In the present invention, the moisture content of the hydrogel or adhesive layer is not particular limited, and may be, for example, 0 mass % or more, 0.5 mass % or more, 1.0 mass % or more, 5.0 mass % or more, 10 mass % or more, 20 mass % or more, 30 mass % or more, 40 mass % or more, 50 mass % or more, 60 mass % or more, 70 mass % or more, or 80 mass % or more, and may be 99 mass % or less, 95 mass % or less, or 90 mass % or less. Note that the moisture content of the adhesive layer can be measured from, for example, the mass loss rate due to heat drying or vacuum drying. Furthermore, the moisture content of the adhesive layer can be determined from the mass ratio of the adhesive component used and the moisture content thereof.

### (Thickness of Adhesive Layer)

The thickness of the adhesive layer is not particularly limited, and may be, for example, in the range of 1.0 μm to 10 mm.

### (Area of Adhesive Layer)

The area of the adhesive layer is not particularly limited and can be appropriately set in accordance with purpose. For example, when the microneedle device is intended to be applied to a part of the face, the area of the adhesive layer, when including the area of the openings, may be, for example, 250 mm² or more, 500 mm² or more, or 1000 mm² or more, and may be 5000 mm² or less, 4000 mm² or less, or 3000 mm² or less.

### <Microneedle Sheet Layer>

In the present invention, the microneedle sheet layer comprises a substrate and a plurality of microneedles which protrude from the substrate.

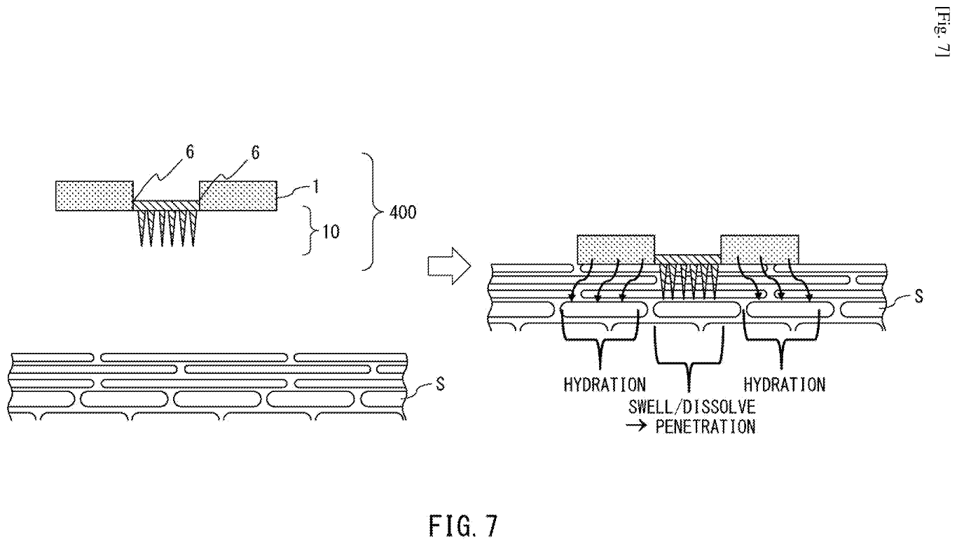

The microneedle sheet layer according to the present invention is arranged so as to at least partially overlap the openings of the adhesive layer described above. Furthermore, from the viewpoint of preventing contact between the microneedle sheet layer and the adhesive layer, it is preferable that the microneedle sheet layer be arranged so as to overlap the openings of the adhesive layer or be arranged inside the openings. When the microneedle sheet layer is arranged inside the openings of the adhesive layer, from the viewpoint of preventing contact between the microneedle sheet layer and the adhesive layer, for example, an adhesive or the like may be arranged on portions where the microneedle sheet layer and the adhesive layer can come into contact with each other (refer to, for example, the adhesive 6 of FIG. 7, which will be described later). Furthermore, even if the microneedle sheet layer is arranged so as to at least partially overlap the openings of the adhesive layer, likewise, an adhesive or the like may be arranged on portions where the microneedle sheet layer and the adhesive layer can come into contact with each other.

### (Microneedles)

The microneedles used in the present invention are formed of a material which can dissolve or swell in vivo. The material which can dissolve or swell in vivo used in the present invention may be any material which exhibits a certain degree of hardness so as to pierce the skin and cause stimulation, and the material itself is not particularly limited. Examples of such materials which can dissolve or swell in vivo include hyaluronic acid, chitosan, maltose, arginate, amylose, agarose and other polysaccharides, carboxymethyl cellulose, hydroxypropyl cellulose and other celluloses, and starches, though the material is not limited thereto. Furthermore, when forming the microneedles, one material which can dissolve or swell in vivo may be used, or a mixture in which two or more types thereof are appropriately mixed may be used.

Among these materials, hyaluronic acid is particularly preferable. Hyaluronic acid is usually used in the form of a salt, and examples of salts include metal salts such as sodium and potassium salts. As the hyaluronic acid, for example, from those with a low molecular weight having an average molecular weight of 10,000 or less as measured by, for example, HPLC to those with a high molecular weight of 800,000 or more can be used. Further, as hyaluronic acid, a mixture of a low molecular weight hyaluronic acid and a high molecular weight hyaluronic acid can be appropriately used.

Note that to the microneedles, there may be added ingredients for water-soluble drugs and cosmetics which have traditionally been used as transdermal preparations, for example, whitening ingredient such as ascorbyl palmitate, kojic acid, rucinol, tranexamic acid, potassium 4-methoxysalicylic acid, pyrimidinylpyrazole compounds, Thuja orientalis extract, oily licorice extract, and vitamin A derivatives; anti-wrinkle ingredients such as retinol, retinoic acid, retinol acetate, retinol palmitate; blood circulation promoting ingredients such as tocopherol acetate, capsaicin, and vanillylamide nonylate; diet ingredients such as raspberry ketone, evening primrose extract, and seaweed extract; antibacterial ingredients such as isopropylmethylphenol, photosensitizer, and zinc oxide; and medicinal ingredients such as vitamins, for example, vitamin D2, vitamin D3, and vitamin K.

In the present invention, the shape of the microneedles is not particularly limited, but conical, truncated conical, and konide-like microneedles are preferable so as to facilitate insertion into the skin and not cause pain at the time of insertion. Konide-type microneedles are so-called “volcanic shape”, wherein the side surfaces of a truncated cone are curved inward.

If the diameters of the bases of the microneedles are small, the microneedles will easily break during insertion into the skin, and if they are large, insertion into the skin will be painful, and thus, a diameter of 0.15 to 1.0 mm, and preferably about 0.15 to 0.5 mm, is appropriate. If the tip diameters thereof are small (pointed), the microneedles will easily break during insertion into the skin, and if they are large, insertion into the skin will be difficult and painful, and thus, a tip diameter of 0.01 to 0.2 mm is suitable.

The heights of the microneedles should be high enough to pierce the skin and cause stimulation, but if they are excessively tall, there is a risk that they may reach the epidermal basement membrane, and they will easily break during insertion into the skin. Thus, heights of the microneedles of, for example, 50 μm to 1000 μm, preferably 100 to 800 μm, and more preferably 300 to 800 μm, are suitable. Furthermore, if the pitch between microneedles becomes short, insertion into the skin becomes difficult, and if it becomes long, the number of microneedles per area decreases, whereby the effect of the present invention may not be exhibited, and thus, a pitch of 0.3 to 1.0 mm is suitable.

Furthermore, the number of microneedles in the microneedle sheet layer is not particularly limited, and may be, for example, 10 to 3000 microneedles per 100 mm² area.

### (Substrate of Microneedle Sheet Layer)

In the present invention, the substrate of the microneedle sheet layer may be the same as or different from the microneedles as long as the microneedles can be formed on the surface thereof. From the viewpoint of more remarkably exhibiting the effect of the present invention, the substrate of the microneedle sheet layer is preferably a material which can dissolve or swell in vivo. Specific examples of materials which can dissolve or swell in vivo are the same as those described above regarding in the “Microneedles” section, and descriptions thereof have been omitted.

The thickness of the substrate of the microneedle sheet layer is not particularly limited, and may be in the range of, for example, 0.01 mm to 2.0 mm.

### (Area of Microneedle Sheet Layer)

The area of the microneedle sheet layer is not particularly limited, and from the viewpoint of exhibiting the affixation effect of the hydrogel layer, the area can appropriately be set in accordance with purpose within a range which is smaller than the area of the hydrogel layer (when the area of the openings is included) described above. For example, when the purpose is for attaching the microneedle device to a part of the face, the area of microneedle sheet layer may be, for example, 200 mm² or more, 500 mm² or more, or 1000 mm² or more, and may be 5000 mm² or less, 4000 mm² or less, or 3000 mm² or less.

### (Electrode Sheet Layer)

The microneedle device of the present invention preferably further comprises an electrode sheet layer on a surface of the hydrogel layer described above on the side opposite the microneedles.

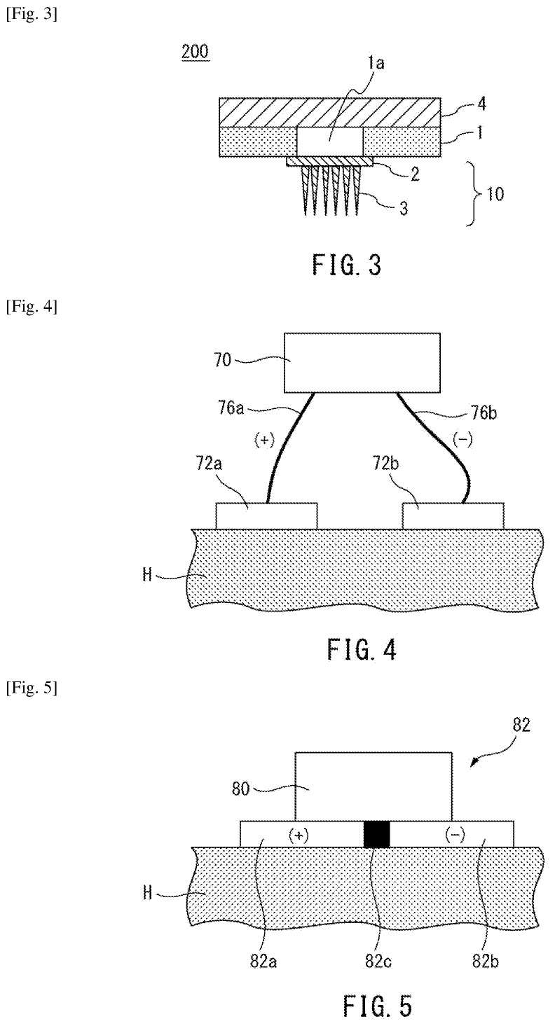

FIG. 3 is a cross-sectional view showing an embodiment of the microneedle device of the present invention including an electrode sheet layer.

As shown in FIG. 3, the microneedle device 200 of the present invention comprises, in this order, an electrode sheet layer 4, an adhesive layer 1 having one or more openings 1a, and a microneedle sheet layer 10 comprising a substrate 2 and a plurality of microneedles 3 which protrude from the substrate 2. Note that though not shown in FIG. 3, the electrode sheet layer 4 may have openings arranged in the same positions as the openings 1a of the adhesive layer 1.

Though not to be bound by theory, a reason why it is preferable for the microneedle device of the present invention to comprise an electrode sheet layer can be considered as follows.

Specifically, due to the electrode sheet layer, a pulse wave having a predetermined frequency can be applied to the skin. This enables skin collagen production, moisturization, and improvement of wrinkles. Thus, it is expected that the collagen production of the skin by the electrode sheet layer and the diffusion of ingredients such as hyaluronic acid into the skin by the microneedle sheet layer are combined, whereby a high beauty effect such as moisturization, elasticity, and elasticity maintenance can be imparted to the skin.

Furthermore, via the adhesive layer of the microneedle device of the present invention, the electrode sheet layer can more easily attach to the skin and a pulse wave can more easily be applied to the skin. In other words, the adhesive layer facilitates exertion of the effect of the electrode sheet layer.

The details of the electrode sheet layer will be described below.

In the present invention, the aspect of the electrode sheet layer is not particularly limited as long as a pulsed current can be applied to the skin. For example, as one aspect, the electrode sheet layer has first and second sheet-like electrodes which are independent of each other, each of the first and second sheet-like electrodes is connected to a current generation unit through a conductive cable, and a pulsed current can be applied to the skin via the adhesive layer. An example thereof is shown in FIG. 4.

In FIG. 4, the electrode sheet layer has first and second sheet-like electrodes 72a, 72b. The first and second sheet-like electrodes 72a, 72b are independent of each other, and can be attached by a user to a preferred part of the skin via the adhesive layer H. The first and second sheet-like electrodes 72a, 72b are electrically connected to a current generation unit 70 by cables 76a, 76b. In the example shown in FIG. 4, the cables 76a, 76b are connected to the first and second sheet-like electrodes 72a, 72b, respectively. The cables 76a, 76b have a common pin jack or pin plug 76c (not illustrated) at the opposite end thereof for connecting to the current generation unit 70.

Though not shown in FIG. 4, the adhesive layer H may have one or more openings. In this case, the sheet-like electrodes 72a, 72b may or may not have openings in the portions overlapping the openings of the adhesive layer H. Furthermore, when the sheet-like electrodes 72a, 72b have one or more openings, each opening may partially overlap an opening of the adhesive layer H, or may completely overlap the opening.

As another aspect, the electrode sheet layer comprises one sheet-like electrode having a first electrode portion, a second electrode portion, and an insulation portion provided between the first and second electrode portions, the one sheet-like electrode has first and second terminals electrically connected to the first electrode portion and the second electrode portion, respectively, and the first and second terminals are connected to the current generation unit, whereby a pulsed current can be applied to the skin via the adhesive layer. An example thereof is shown in FIG. 5.

In FIG. 5, the electrode sheet layer has a single sheet-like electrode 82. The sheet-like electrode 82 has a first electrode portion 82a and a second electrode portion 82b, and an insulation portion 82c is provided therebetween. The first and second electrode portions 82a, 82b and the insulation portion 82c are formed as one sheet-like member. The sheet-like electrode 82 has first and second electrode side terminals (not illustrated) connected to the first and second electrode portions 82a, 82b, respectively. The current generation unit 80 includes first and second power supply side terminals (not illustrated) that can be hooked or snapped to and separated from the first and second electrode side terminals. By coupling the first and second electrode-side terminals to the first and second electrode-side terminals, the sheet-like electrode 82 is connected to the current generation unit 80 without the use of a cable. At this time, the current generation unit 80 is attached to the sheet-like electrode 82.

Though not shown in FIG. 5, the adhesive layer H may have one or more openings, and in this case, the sheet-like electrodes 82a and 82b may or may not have openings in the portions overlapping the openings of the adhesive layer H. Furthermore, when the sheet-like electrodes 82a and 82b have one or more openings, each opening may partially overlap an opening of the adhesive layer H, or may completely overlap the opening.

In the present invention, the material of the electrode sheet layer is not particularly limited, and may be, for example, a conductive polymer, carbon black, resin, dielectric elastomer actuator (DEA), IPMC actuator, or PVC gel having flexibility so as to enable the shape thereof to match the concavities and convexities of the skin. Furthermore, the material may be thin so that it can be attached to the skin of the user, it may be transparent so that the attachment is inconspicuous, and it may match the color of the skin of the user. The electrode sheet layer may be used repeatedly or may be disposable.

Furthermore, as described above, the electrode sheet layer may be used as the substrate of the adhesive layer. In this case, a laminate body of the electrode sheet layer and the hydrogel may be regarded as the adhesive layer, and one or more openings may be provided therein.

### <Support Layer>

The microneedle device of the present invention may further comprise a support layer. This support layer may be arranged, for example, in the openings of the adhesive layer. As a result, the microneedle sheet layer is supported by the support layer. By including this support layer, the microneedle sheet layer can easily be arranged so as to overlap the openings of the adhesive layer, and specifically, a structure in which the microneedle sheet layer and the adhesive layer do not contact each other can easily be obtained.

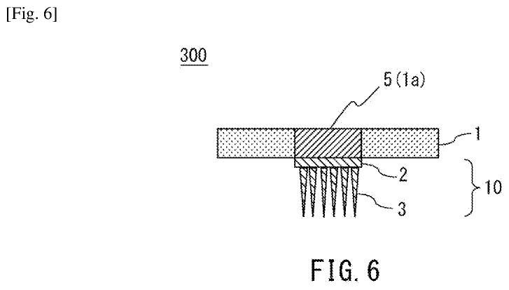

For example, FIG. 6 is a cross-sectional view showing an embodiment of the mi- croneedle device of the present invention including a support layer.

As shown in FIG. 6, the microneedle device 300 of the present invention comprises an adhesive layer 1 having one or more openings 1a, a microneedle sheet layer 10 comprising a substrate 2 and a plurality of microneedles 3 which protrude from the substrate 2, and a support layer 5 arranged in openings 1a of the adhesive layer 1. The microneedle sheet layer 10 is supported by the support layer 5.

The material constituting the support layer is not particularly limited, and may be, for example, a resin which does not dissolve or swell in vivo and which does not affect the living body. More specifically, examples of materials which can constitute the support layer include polymethylmethacrylate, cellulose acetate, ethylcellulose, polyethylene resins, polypropylene resins, ethylene-propylene copolymers, ethylene-vinyl acetate copolymers, vinyl chloride resins, vinylidene chloride resins, vinyl acetate-vinyl chloride copolymers, polyamide resins, polyester resins, ABS resins, SIS resins, SEBS resins, urethane resins, silicon resins, and aluminum.

The thickness of the support layer is not particularly limited, and may be, for example, the same thickness as the adhesive layer.

### <Other Members>

The microneedle device of the present invention may further comprise other members in addition to the members described above as long as the effect of the present invention is not impaired thereby.

The microneedle device of the present invention may further comprise, for example, a protective sheet layer. The protective sheet layer can be used for covering the exposed portions of the adhesive layer. The protective sheet layer may be peeled off immediately before the microneedle device of the present invention is applied to the skin.

The material constituting the protective sheet layer is not particularly limited, and may be, for example, a film such as a polyethylene, polypropylene, or polyester film.

### <Production Method>

The method for the production the microneedle device of the present invention is not particularly limited, and for example, if necessary, members may be each prepared and may be assembled.

### (Preparation of Adhesive Layer)

The adhesive layer according to the present invention can be obtained by forming one or more openings in a commercially available product or a product obtained by applying an adhesive component to a substrate.

Furthermore, when an electrode sheet layer is used as the substrate of the adhesive layer, it can be obtained by applying the adhesive on the electrode sheet layer and forming one or more openings therein. Further, it can also be obtained by forming one or more openings using a commercially available electrode sheet layer provided with an adhesive layer.

### (Preparation of Microneedle Sheet Layer)

The microneedle sheet layer according to the present invention can be obtained by, for example, the following method.

### Method (1)

Method (1) is a method in which an aqueous solution of a material primarily composed of a microneedle-forming material such as hyaluronic acid, to which a medicinal ingredient or a cosmetic ingredient has been added as needed, is cast into a mold formed in the shape of the microneedles, the moisture is evaporated and drying is carried out at room temperature or by heating, and after laminating the substrate, the mold is peeled away and the microneedles are transferred onto the substrate.

### Method (2)

Method (2) is a method in which a layer of the aqueous solution described above is cast on the mold surface of method (1) described above for formation, the moisture is evaporated at room temperature or heated to evaporate, and the mold is then peeled away. In this method, a microneedle sheet layer in which both the substrate and the microneedles are composed of a material primarily composed of a microneedle-forming material, to which a medicinal ingredient or a cosmetic ingredient has been added as needed, is obtained.

### Method (3)

Method (3) is a method in which an aqueous solution of a material primarily composed of microneedle-forming material, to which a medicinal ingredient or a cosmetic ingredient has been added as needed, is injection-molded into the shape of microneedles on a substrate, and then dried at room temperature or by heating to evaporate the moisture.

### <Applications>

The microneedle device of the present invention is particularly suitable for skincare. Here, examples of skincare include but are not limited to moisturizing, whitening, wrinkle improvement, spot improvement, color unevenness improvement, sagging improvement, dullness improvement, dark circle improvement, pore improvement, acne improvement, and impartment of tension and elasticity.

Furthermore, the microneedle device of the present invention may be packaged as a single unit or as a plurality of units for skincare use. The packaging is preferably vacuum packaging in which the microneedle device of the present invention is sealed and the interior of the packaging is depressurized.

### Beautification Method

The present invention also provides a beautification method.

One aspect of the beautification method according to the present invention is shown below.

Specifically, a first beautification method of the present invention provides:
a beautification method using the microneedle device of the present invention, the method comprising the step of:
attaching and adhering the microneedle side of the microneedle device to skin, whereby at least a part of a constituent material of the microneedles penetrates the skin.

For example, FIG. 7 is a pattern diagram for detailing an embodiment of the first beautification method of the present invention. As shown in FIG. 7, the beautification method of the present invention uses the microneedle device 400. As shown on the right side of the drawing of FIG. 7, the first beautification method includes attaching and adhering the microneedle side of the microneedle device 400 to the skin S, whereby at least a part of a constituent material of the microneedles penetrates the skin. At this time, when the adhesive layer 1 contains moisture, for example, when the adhesive layer is a hydrogel layer, the skin S can be hydrated from the periphery of the microneedles.

Note that in the microneedle device 400, though the microneedle sheet layer 100 is configured to be contained in the openings of the adhesive layer 1, an adhesive 6 is provided between the microneedle sheet layer 100 and the adhesive layer 1. The adhesive 6 affixes the microneedle sheet layer 100 and the adhesive layer 1 and prevents direct contact therebetween.

Another aspect of the beautification method according to the present invention is shown below.

Specifically, a second beautification method of the present invention provides:
a beautification method using the microneedle device according to the present invention, the method comprising the steps of:
attaching and adhering the microneedle side of the microneedle device to skin, whereby at least a part of a constituent material of the microneedles penetrates the skin, and
applying a pulsed current to the skin via the electrode sheet layer.

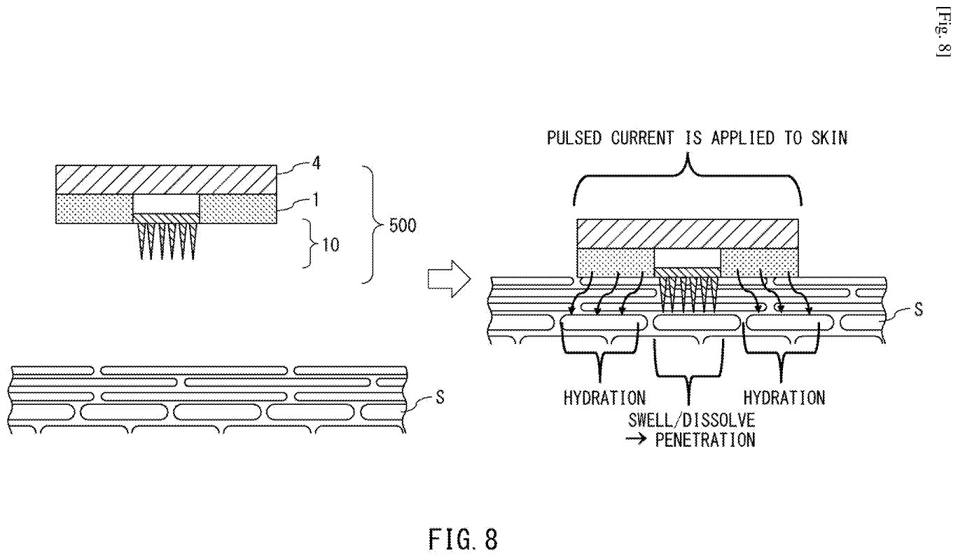

For example, FIG. 8 is a pattern diagram for detailing an embodiment of the second beautification method of the present invention. As shown in FIG. 8, the beautification method of the present invention uses the microneedle device 500. As shown on the right side of the drawing of FIG. 8, the second beautification method includes attaching and adhering the microneedle side of the microneedle device 500 to the skin S, whereby at least a part of a constituent material of the microneedles penetrates the skin, and applying a pulsed current to the skin via the electrode sheet layer 4. At this time, when the adhesive layer 1 contains moisture, for example, when the adhesive layer is a hydrogel layer, the skin S can be hydrated from the periphery of the microneedles.

Note that the microneedle device 500 is identical to the microneedle device 400, except that an electrode sheet layer 4 is further included on the adhesive layer 1 of the microneedle device 400 of FIG. 7 on the side opposite the microneedles, and thus, description thereof has been omitted.

In the second beautification method of the present invention, the pulsed current may have a frequency in the range of, for example, 100 to 5000 Hz, more preferably 200 to 5000 Hz, and further preferably 500 to 2000 Hz. Furthermore, the current value of the pulsed current can be arbitrarily selected, for example, 100 μA to 1000 μA, 150 μA to 1000 μA, or 200 μA to 500 μA. The current value is preferably 200 μA to 500 μA. Since a current value in such a range is equivalent to the current value of human skin, there is little adverse effect on skin. The voltage can be arbitrarily selected from 1.0 V to 10.0 V, 5.0 V to 10.0 V, or 1.0 V to 5.0 V within a range which does not adversely affect the skin. The pulse wave and voltage waveform can be arbitrarily set, such as a pulse wave, a square wave, a sine wave, a triangular wave, or a saw wave.

In the beautification method of the present invention, the time during which at least a part of the constituent material of the microneedles penetrates the skin is not particularly limited, and may be, for example, 1 hour or more, 3 hours or more, 6 hours or more, or 12 hours or more, and may be 24 hours or less or 12 hours or less.

In the second beautification method of the present invention, the time during which a pulsed current is applied to the skin is not particularly limited, and may be, for example, 1 minute or more, 5 minutes or more, 10 minutes or more, 20 minutes or more, or 40 minutes or more, and may be 8 hours or less, 4 hours or less, 3 hours or less, 2 hours or less, or 1 hour or less.

Furthermore, the frequency at which the beautification method of the present invention is performed is not particularly limited, and it may be performed once daily, every two days, three days, four days, five days, six days, or seven days, continuously or intermittently on a regular or irregular basis once every one, two, three, or four weeks.

### EXAMPLES

The present invention will be described in more detail below with reference to the Examples, but the present invention is not limited thereto.

### Examples 1 and 2 and Comparative Example 1

### Example 1

In Example 1, a conductive carbon sheet was used as the electrode sheet layer. An adhesive layer containing moisture was bonded to this electrode sheet layer to form a 20 mm×20 mm square laminate of an electrode sheet and an adhesive layer. A circular opening having a diameter of 8 mm was then provided in the center of the laminate. Circular microneedles having a diameter of 9 mm and an area of 64 mm² were prepared, a ring-shaped adhesive was attached to an area 2 mm from the outer circumference of the microneedle sheet layer, and the microneedles were laminated on the laminate via an adhesive so that the openings of the microneedle sheet layer and the laminate overlapped, whereby the microneedle device 1 was produced.

A microneedle sheet layer (area: 64 mm2) having 138 microneedles formed of hyaluronic acid having a height of 200 um, as the microneedles, and containing 2% by mass of a fluorescent agent was used.

### Example 2>

In Example 2, a microneedle device 2 was produced in the same manner as Example 1 except that the electrode sheet layer was not provided.

### Comparative Example 1

In Comparative Example 1, the microneedles of Example 1 were used as a microneedle device 3 without an electrode sheet layer and an adhesive layer.

### Penetration Strength Measurement

Using the microneedle devices of Examples 1 and 2 and Comparative Example 1, the hyaluronic acid penetration strength when the devices were applied to human skin was examined.

More specifically, in Example 1, the microneedle device 1 was attached to human skin, the microneedle device 1 and the skin were firmly secured using an affixation device, and a minute current of a pulse wave of 5 V, 250 μA, and 500 Hz was applied to the skin via the electrode sheet for 1 hour. Thereafter, the affixation device was removed, and the minute current was continuously applied to the skin for 1.5 hours.

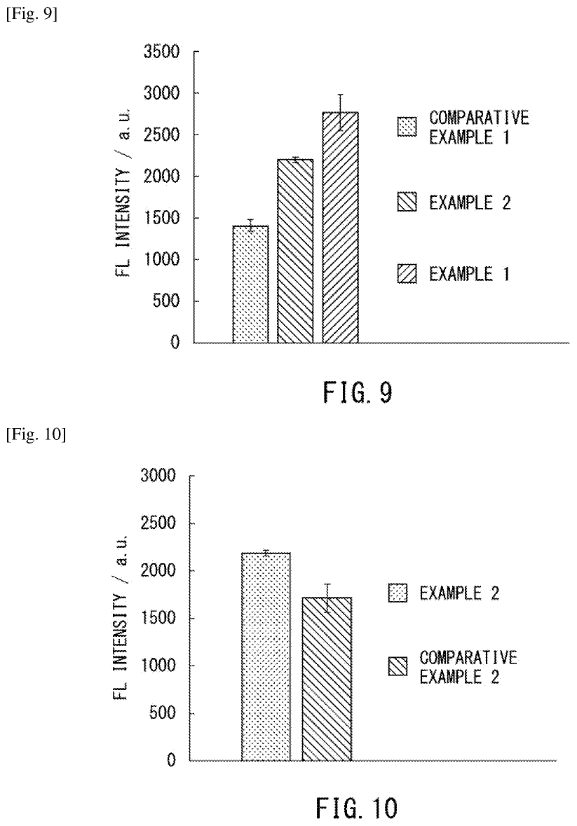

Thereafter, the microneedle device 1 was removed, the surface of the skin where the microneedles were applied was cleaned, and a portion of the skin was cut into a circle having a diameter of 8 mm with a cutter. The obtained skin was placed in 400 μL of methanol and subjected to sonication to extract the fluorescent dye. The fluorescence intensity (Ex: 488 nm, Em: 515 nm) of 200 μL of the extracted sample was then measured with a spectrophotometric system. The measurement result is evaluated as the penetration strength of the microneedle device 1 and is shown in FIG. 9.

In Example 2, after the microneedle device 2 was applied to human skin for 2.5 hours, the fluorescence intensity was measured by the same operation as Example 1. The results of the obtained penetration strength are shown in FIG. 9.

In Comparative Example 1, after the microneedle device 3 was applied to human skin for 2.5 hours, the fluorescence intensity was measured by the same operation as Example 1. The results of the obtained penetration strength are shown in FIG. 9.

As is clear from FIG. 9, it could be understood that both microneedle devices 1 and 2 of Examples 1 and 2 had higher hyaluronic acid penetration strength as compared to Comparative Example 1. In particular, it could be understood that the microneedle device 1 of Example 1 had the highest hyaluronic acid penetration strength.

### Comparative Example 2

In Comparative Example 2, a microneedle device 4 was produced in the same manner as Example 2 except that the adhesive layer was not provided with an opening.

After the microneedle device 4 was applied to human skin for 2.5 hours, the fluorescence intensity was measured by the same operation as Example 1. The results of the obtained penetration strength are shown in FIG. 10.

The results of Example 2 are also shown in FIG. 10 for comparison with Comparative Example 2.

As is clear from the results of FIG. 10, it could be understood that the microneedle device 2 (Example 2), which had an opening in the adhesive layer, had a higher hyaluronic acid penetration strength than the microneedle device 4 (Comparative Example 2), which did not have an opening in the adhesive layer.

### REFERENCE SIGNS LIST

1, 1A, 1B, 1C, 1D, 1E, H adhesive layer
1a, 1b, 1c, 1d, le opening
2 substrate of microneedle sheet layer
3 microneedle
4 electrode sheet layer
5 support layer
6 adhesive
10 microneedle sheet layer
70, 80 current generation unit
72a, 72b, 82 sheet-like electrode
76a, 76b cable
82a first electrode portion
82b second electrode portion
82c insulation portion
100, 200, 300, 400, 500 microneedle device
S skin

1. A microneedle device, comprising an adhesive layer and a microneedle sheet layer,
wherein the adhesive layer has one or more openings,
wherein the microneedle sheet layer has a substrate and a plurality of microneedles which protrude from the substrate,
wherein the substrate and the microneedles are formed from a material which dissolves or swells in vivo, and
wherein the microneedle sheet layer is arranged so as to at least partially overlap the openings of the adhesive layer.

2. The microneedle device according to claim 1, further comprising an electrode sheet layer on a surface of the adhesive layer on a side opposite the microneedles.

3. The microneedle device according to claim 1, wherein the microneedle sheet layer is arranged so as to overlap the openings of the adhesive layer or inside the openings.

4. The microneedle device according to claim 1, further comprising a support layer,
wherein the support layer is arranged in the openings of the adhesive layer, and
wherein the microneedle sheet layer is supported on the support layer.

5. The microneedle device according to claim 1, wherein the material which dissolves or swells in vivo is hyaluronic acid.

6. The microneedle device according to claim 1, for skincare use.

7. A beautification method using the microneedle device according to claim 1, the method comprising:
attaching and adhering the microneedle side of the microneedle device to skin, whereby at least a part of a constituent material of the microneedles penetrates the skin.

8. A beautification method using the microneedle device according to claim 2, the method comprising:
attaching and adhering the microneedle side of the microneedle device to skin, whereby at least a part of a constituent material of the microneedles penetrates the skin, and applying a pulsed current to the skin via the electrode sheet layer.
